(19) United States
(12) Patent Application Publication
(10) Pub. No.: US 2014/0213881 A1
(43) Pub. Date: Jul. 31, 2014

(54) MAGNETICALLY CONNECTED ELECTRODE FOR MEASURING PHYSIOLOGICAL SIGNALS

(71) Applicant: Perminova Inc., La Jolla, CA (US)

(72) Inventors: Matt Banet, Kihei, HI (US); Susan Pede, Encinitas, CA (US); Marshal Dhillon, San Diego, CA (US); Drew Terry, San Diego, CA (US)

(73) Assignee: Perminova Inc., La Jolla, CA (US)

(21) Appl. No.: 14/167,519

(22) Filed: Jan. 29, 2014

Related U.S. Application Data
(60) Provisional application No. 61/757,982, filed on Jan. 29, 2013.

(51) Int. Cl. A61B 5/00; A61B 5/053; A61B 5/0408

(52) U.S. Cl. CPC A61B 5/6802; A61B 5/0408; A61B 5/053
USPC 600/393

(57) ABSTRACT

The invention provides an electrode and associated electrode holder that are used for physiological measurements, e.g. measurements of signals that can be processed to generate ECG and TBI waveforms. The electrode and electrode holder connect to each other using a magnetic interface. In embodiments, for example, the magnetic interface includes oppositely polled magnets integrated in both the electrode and electrode holder. The magnets are typically rare earth magnets coated with a thin, electrically conductive metal film. This way, when the magnets come in contact with each other, the metal films touch to form both a mechanical and electrical connection. Thus the magnetic interface can replace conventional mechanisms used to connect rivet-based electrodes to leads, which are typically used to secure electrodes for physiological measurements.

### CROSS REFERENCES TO RELATED APPLICATIONS

This application claims the benefit of U.S. Provisional Application No. 61/757,982, filed Jan. 29, 2013, which is hereby incorporated in its entirety including all tables, figures, and claims.

### BACKGROUND OF THE INVENTION

1. Field of the Invention

The present invention relates to electrodes and sensors that use them to measure physiological signals from patients.

2. Description of the Related Art

Medical devices can measure time-dependent electrocardiograms (ECG) and thoracic bioimpedance (TBI) waveforms from patients. Such devices typically connect to disposable electrodes that adhere to the patient's skin and measure bioelectric signals. Analog circuits within the device process the signals to generate the waveform, which with further analysis yields parameters such as heart rate (HR), stroke volume (SV), and cardiac output (CO). Most conventional electrodes used in this capacity include an adhesive, conductive gel. Connected to the gel is a metal component that features a flat surface coated with a silver/silver chloride (Ag/AgCl) film, and a metal rivet that mates to an electrical lead. During use, the conductive gel sticks to the patient's skin, and the metal rivet snaps into the electrical lead. The lead, which typically terminates a long cable, then plugs into the device making the measurement. Working in concert, the conductive gel and Ag/AgCl film sense bioelectric signals from the patient, which travel through the metal component into the electrical lead, and finally to the device's analog circuit for processing.

Devices that measure ECG and TBI waveforms are often used to characterize patients suffering from congestive heart failure (CHF). CHF occurs when the heart is unable to sufficiently pump and distribute blood to meet the body's needs. CHF is typically preceded by an increase of fluid in the thoracic cavity, and can by characterized by shortness of breath, swelling of the legs and other appendages, and intolerance to exercise. It affects nearly 5.3 M Americans and has an accompanying cost of somewhere between $30-50 B, with roughly $17 B attributed to hospital readmissions. Such events are particularly expensive to hospitals, as readmissions occurring within a 30-day period are not reimbursable by Medicare or private insurance as of October 2012.

In medical centers, CHF is typically detected using Doppler/ultrasound, which measures parameters such as SV, CO, and ejection fraction (EF). Gradual weight gain measured with a simple scale is one method to indicate CHF in the home environment. However, this parameter is typically not sensitive enough to detect the early onset of CHF, a particularly important time when the condition may be ameliorated by a change in medication or diet.

SV is the mathematical difference between left ventricular end-diastolic volume (EDV) and end-systolic volume (ESV), and represents the volume of blood ejected by the left ventricle with each heartbeat; a typical value is about 80 mL. EF relates to EDV and ESV as described below in Eq. 1, with a typical value for healthy individuals being about 50-65%, and an ejection fraction of less than 40% indicating systolic heart failure.

$$E F = \frac{S V}{E D V} = \frac{E D V - E S V}{E D V} \qquad ( 1 )$$

CO is the average, time-dependent volume of blood ejected from the left ventricle into the aorta and, informally, indicates how efficiently a patient's heart pumps blood through their arterial tree; a typical value is about 5 L/min. CO is the product of HR and SV, i.e.:

CO=SV×HR (2)

CHF patients, in particular those suffering from systolic heart failure, may receive implanted devices, such as pacemakers and/or implantable cardioverter-defibrillators, to increase EF and subsequent blood flow throughout the body. These devices also include technologies called ‘OptiVol’ (from Medtronic) or ‘CorVue’ (St. Jude) that use circuitry and algorithms within the implanted device to measure the electrical impedance between different leads of the pacemaker. As thoracic fluid increases in the CHF patient, the impedance typically is reduced. Thus this parameter, when read by an interrogating device placed outside the patient's body, can indicate the onset of heart failure.

Corventis Inc. has developed the AVIVO Mobile Patient Management (MPM) System to characterize ambulatory CHF patients. AVIVO is typically used over a 7-day period, during which it provides continual insight into a patient's physiological status by steadily collecting data and wirelessly transmitting it through a small handheld device to a central server for analysis and review. The system consists of three parts: 1) The PiiX sensor, a patient-worn adhesive device that resembles a large (approximately 15″ long) bandage and measures fluid status, ECG waveforms, HR, respiration rate, patient activity, and posture; 2) The zLink Mobile Transmitter, a small, handheld device that receives information from the Piix sensor and then transmits data wirelessly to a remote server via cellular technology; and 3) the Corventis Monitoring Center, where data are collected and analyzed. Technicians staff the Monitoring Center, review the incoming data, and in response generate clinical reports made available to prescribing physicians by way of a web-based user interface.

In some cases, physicians can prescribe ambulatory monitors to CHF patients. These systems measure time-dependent ECG waveforms, from which HR and information related to arrhythmias and other cardiac properties are extracted. They characterize ambulatory patients over short periods (e.g. 24-48 hours) using ‘holier’ monitors, or over longer periods (e.g. 1-3 weeks) using cardiac event monitors. Conventional holter or event monitors typically include a collection of chest-worn ECG electrodes (typically 3 or 5), an ECG circuit that collects analog signals from the ECG electrodes and converts these into multi-lead ECG waveforms; a processing unit then analyzes the ECG waveforms to determine cardiac information. Typically the patient wears the entire system on their body. Some modern ECG-monitoring systems include wireless capabilities that transmit ECG waveforms and other numerical data through a cellular interface to an Internet-based system, where they are further analyzed to generate, for example, reports describing the patient's cardiac rhythm. In less sophisticated systems, the ECG monitoring system is worn by the patient, and then returned to a company that downloads all relevant information into a computer, which then analyzes it to generate the report. The report, for example, may be imported into the patient's electronic medical record (EMR). The EMR avails the report to cardiologists or other clinicians, who then use it to help characterize the patient.

Measuring CO and SV in a continuous, non-invasive manner with high clinical accuracy has often been considered a ‘holy grail’ of medical-device monitoring. Most existing techniques in this field require in-dwelling catheters, which in turn can lead to complications with the patient, are inherently inaccurate in the critically ill, and require a specially trained operator. For example, current ‘gold standards’ for this measurement are thermodilution cardiac output (TDCO) and the Fick Oxygen Principal (Fick). However both TDCO and Fick are highly invasive techniques that can cause infection and other complications, even in carefully controlled hospital environments. In TDCO, a pulmonary artery catheter (PAC), also known as a Swan-Ganz catheter, is typically inserted into the right portion of the patient's heart. Procedurally a bolus (typically 10 ml) of glucose or saline that is cooled to a known temperature is injected through the PAC. A temperature-measuring device within the PAC, located a known distance away (typically 6-10 cm) from where fluid is injected, measures the progressively increasing temperature of the diluted blood. CO is then estimated from a measured time-temperature curve, called the ‘thermodilution curve’. The larger the area under this curve, the lower the cardiac output Likewise, the smaller the area under the curve implies a shorter transit time for the cold bolus to dissipate, hence a higher CO.

Fick involves calculating oxygen consumed and disseminated throughout the patient's blood over a given time period. An algorithm associated with the technique incorporates consumption of oxygen as measured with a spirometer with the difference in oxygen content of centralized blood measured from a PAC and oxygen content of peripheral arterial blood measured from an in-dwelling cannula.

Both TD and Fick typically measure CO with accuracies between about 0.5-1.0 l/min, or about +/−20% in the critically ill.

Several non-invasive techniques for measuring CO and SV have been developed with the hope of curing the deficiencies of Fick and TD. For example, Doppler-based ultrasonic echo (Doppler/ultrasound) measures blood velocity using the well-known Doppler shift, and has shown reasonable accuracy compared to more invasive methods. But both two and three-dimensional versions of this technique require a specially trained human operator, and are thus, with the exception of the esophageal Doppler technique, impractical for continuous measurements. CO and SV can also be measured with techniques that rely on adhesive electrodes placed on the patient's torso that inject and then collect a low-amperage, high-frequency modulated electrical current. These techniques, based on electrical bioimpedance and called ‘impedance cardiography’ (ICG), ‘electrical cardiometry velocimetry’ (ECV), and ‘bioreactance’ (BR), measure a time-dependent electrical waveform that is modulated by the flow of blood through the patient's thorax. Blood is a good electrical conductor, and when pumped by the heart can further modulate the current injected by these techniques in a manner sensitive to the patient's CO. During a measurement, ICG, ECV, and BR each extract properties called left ventricular ejection time (LVET) and pre-injection period (PEP) from time-dependent ICG and ECG waveforms. A processer then analyzes the waveform with an empirical mathematical equation, shown below in Eq. 3, to estimate SV. CO is then determined from the product of SV and HR, as described above in Eq. 2.

ICG, ECV, and BR all represent a continuous, non-invasive alternative for measuring CO/SV, and in theory can be conducted with an inexpensive system and no specially trained operator. But the medical community has not embraced such methods, despite the fact that clinical studies have shown them to be effective with some patient populations. In 1992, for example, an analysis by Fuller et al. analyzed data from 75 published studies describing the correlation between ICG and TD/Fick (Fuller et al., The validity of cardiac output measurement by thoracic impedance: a meta-analysis; Clinical Investigative Medicine; 15: 103-112 (1992)). The study concluded using a meta analysis wherein, in 28 of these trials, ICG displayed a correlation of between r=0.80-0.83 against TDCO, dye dilution and Fick CO. Patients classified as critically ill, e.g. those suffering from acute myocardial infarction, sepsis, and excessive lung fluids, yielded worse results. Further impeding commercial acceptance of these techniques is the tendency of ICG monitors to be relatively bulky and similar in both size and complexity to conventional vital signs monitors. This means two large and expensive pieces of monitoring equipment may need to be located bedside in order to monitor a patient's vital signs and CO/SV. For this and other reasons, impedance-based measurements of CO have not achieved widespread commercial success.

### SUMMARY OF THE INVENTION

The current invention provides a simple, low-cost electrode that features a magnetic interface in place of the metal rivet used in conventional electrodes. The electrode measures signals that, when processed, yield ECG and TBI waveforms, from which parameters such as HR, SV, and CO can be calculated. The electrode connects through the magnetic interface to deliver bioelectric signals to analog and digital circuits within a sensor, where they are processed to generate the above-described parameters. The sensor can also measure other parameters such as arrhythmias, temperature, location, and motion/posture/activity level.

In embodiments, the above-mentioned parameters can be used to characterize patients suffering from CHF and other conditions. The sensor, which in embodiments is shaped like a conventional necklace, is particularly designed for ambulatory patients: with this form factor, it can be easily draped around a patient's neck, where it then makes the above-described measurements during the patient's day-to-day activities. Using a short-range wireless radio, the sensor transmits data to the patient's cellular telephone, which then processes and retransmits the data over cellular networks to a web-based system. The web-based system generates reports for supervising clinicians, who can then adjust the patient's diet, exercise, and medication regime to prevent the onset of CHF.

The sensor features a miniaturized impedance-measuring system, described in detail below, that is built into the necklace form factor. Electrodes described herein connect to opposing strands of the necklace through separate magnetic interfaces. This system measures a time-dependent, TBI waveform having two components: an AC component that features a heartbeat-induced pulse, and a DC component that varies with impedance within the patient's chest. With processing, the AC component yields HR, SV, and CO, while the DC component yields thoracic fluid levels. Accompanying this system is a collection of algorithms that perform signal processing and account for the patient's motion, posture and activity level, as measured with an internal accelerometer, to improve the calculations for all hemodynamic measurements. Compensation of motion is particularly important since measurements are typically made from ambulatory patients. Also within the necklace is a medical-grade ECG system that measures single-lead ECG waveform with the magnetically connected electrodes, along with accompanying values of HR and cardiac arrhythmias. The system can also analyze other components of the ECG waveforms, which include: i) a QRS complex; ii) a P-wave; iii) a T-wave; iv) a U-wave; v) a PR interval; vi) a QRS interval; vii) a QT interval; viii) a PR segment; and ix) an ST segment. The temporal or amplitude-related features of these components may vary over time, and thus the algorithmic-based tools within the system, or software associated with the algorithm-based tools, can analyze the time-dependent evolution of each of these components. In particular, algorithmic-based tools that perform numerical fitting, mathematical modeling, or pattern recognition may be deployed to determine the components and their temporal and amplitude characteristics for any given heartbeat recorded by the system.

Each of the above-mentioned components corresponds to a different feature of the patient's cardiac system, and thus analysis of them according to the invention may determine or predict the onset of CHF.

The electrode and electrode holder connect to each other using a magnetic interface, i.e. a magnetic field. In embodiments, for example, the magnetic interface includes oppositely polled magnets integrated in both the electrode and electrode holder. The magnets are typically rare earth magnets coated with a thin, electrically conductive metal film. This way, when the magnets are drawn to each other through the resultant magnetic field, the metal films touch to form both a mechanical and electrical connection. Thus the magnetic interface can replace conventional mechanisms used to connect rivet-based electrodes to leads, which are typically used to secure electrodes for physiological measurements.

In one aspect, the invention provides a system for making a physiological measurement that includes: i) an electrode with at least one electrode region having a metal film in electrical contact with a first magnet; and ii) an electrode holder with at least one conductive region having an electrical trace in electrical contact with a second magnet. The first and second magnets are orientated (e.g., positioned so that their poles are opposing) to generate a magnetic field that causes them to mechanically connect when held proximal to each other. Additionally this electrically connects the metal trace to the electrode's conductive region, thus facilitating the physiological measurement.

In this and other embodiments, one of the magnets can be replaced with a magnetically active material, such as metals containing iron.

In another aspect, the invention provides an electrode for measuring bioimpedance signals. The electrode includes two electrode regions, both having: i) a conductive gel; ii) an Ag/AgCl film comprising Ag/AgCl in electrical contact with the conductive gel; iii) a metal film in electrical contact with the first Ag/AgCl film; and iv) a magnet in electrical contact with the first metal film. The first electrode region makes an electrical connection to a first electrical circuit that injects an electrical current through it and then into the patient's body. The second electrode region makes an electrical connection to a second electrical circuit that measures a voltage related to the injected current and the patient's bioimpedance. In embodiments, the electrode connects to sensors that process the voltage to measure bioimpedance signals. For example, the electrode's magnetic interface can hold the sensor on the patient's body.

In another aspect, the invention provides a sensor configured to be worn on a patient's body and measure a physiological property. The sensor includes an electrode similar to that described above, and an electrode holder that mechanically and electrically connects to the electrode to receive the physiological signal. This component includes: i) an electrical trace; and ii) a second magnet in electrical contact with the electrical trace and oriented to connect to the first magnet in the electrode. An analog circuit is in electrical contact with the electrical trace, and features electrical components (e.g. amplifiers, resistors, capacitors, and other components pieced together to form an electrical circuit). The analog circuit receives the physiological signal from the electrode holder and, in response, generates a processed physiological signal. Finally, the system includes a digital circuit, in electrical contact with the analog circuit, with: i) an analog-to-digital converter that receives the processed physiological signal and, in response, digitizes it to generate a digital physiological signal; and ii) a microprocessor that receives the digital physiological signal and, in response, processes it to generate the physiological property.

In another aspect, the invention provides a sensor having magnetically connected electrodes that are configured to be worn around the neck of a patient and measure a physiological property. The sensor features first and second electrodes connected to the necklace-shaped sensor in such a way (e.g. on opposing strands of the necklace) so that the first electrode adheres to a first side of the patient's body, and the second electrode adheres to a second, opposing side of the body. Both electrodes, during use, are proximal to the patient's neck. They have electrical/mechanical properties similar to those described above, and measure first and second physiological signals from the patient. During a measurement, they attach, respectively, to first and second electrode holders that both include: i) an electrical trace; and ii) a magnet in electrical contact with the electrical trace and oriented to connect to the magnet in the mated electrode. The electrode holders receive first and second physiological signals, and pass them to an electrically connected analog circuit that, in response, generates a processed physiological signal. The system also includes a digital circuit in electrical contact with the analog circuit. It includes: i) an analog-to-digital converter that receives the processed physiological signal and, in response, digitizes it to generate a digital physiological signal; and ii) a microprocessor that receives the digital physiological signal and, in response, processes it to generate the physiological property. For example, the physiological property could be an ECG waveform, TBI waveform, HR, SV, CO, or vital sign value, or fluid levels within the patient's thoracic cavity.

In yet another aspect, the invention provides a method for measuring a bioimpedance signal from a patient. The method features the following steps: i) contacting a first region of the patient's body with a first and second magnetically connected electrode, each having a mechanical/electrical configuration similar to that described above; ii) injecting an electrical current into the patient through a magnet in the first electrode; iii) measuring a signal from the second magnet to generate a voltage related to a product of the electrical current injected through the first electrode and a bioimpedance of the patient; and iv) processing the voltage to generate the bioimpedance signal.

The invention has many advantages. In general, electrodes and electrode holders having the magnetic interface described above can be easily connected to each other to make a physiological measurement. With this system a clinician does not need to apply force to connect the two components during a measurement: they can simply be held proximal to each other, and then the magnetic field between the two components will force them together. For example, in one use case, the clinician can simply hold the electrode holder near the electrode, and the magnetic interface causes the two components to rapidly ‘snap’ together to form an electrical/mechanical connection. In another use case, the electrode is first adhered to a patient's skin, and then the electrode holder is held nearby, causing it to snap into the electrode. Other use cases are, of course, possible. As described above, in all cases one magnet in either the electrode or electrode holder can be replaced with a magnetically active material, e.g. a material containing iron.

Magnetically connected electrodes and electrode holders work particularly well with the necklace-shaped sensor of the invention. One embodiment of this sensor is designed for at-home measurements of patients with CHF or other cardiac diseases. Often, such patients are elderly, and may lack the manual dexterity to connect multiple, conventional electrodes and leads having metal snaps and rivets. With the system of the invention, the patient only need to hold the electrodes and electrode holders close to one another; the magnetic interface connects these components without any effort from the patient. The system is particularly advantageous for multi-part electrodes, which would otherwise require the patient to press together the rivets of multiple electrodes and their corresponding snaps.

These and other advantages will be apparent from the following detailed description, and from the claims.

### BRIEF DESCRIPTION OF THE DRAWINGS

FIG. 1A-E are photographs of, respectively: i) a 3-part electrode featuring a magnetic interface; ii) a mated, 3-part electrode holder featuring an oppositely polled magnetic interface; iii) the electrode and electrode holder held proximal to one another; iv) the electrode and electrode holder connected by a magnetic field between the electrode and electrode holder; and v) the Ag/AgCl-coated metal film on the backside of the electrode, which is secured to the electrode holder by the magnetic interface;

FIG. 2 shows a three-dimensional image of a necklace-shaped sensor that uses the magnetically connected electrode of FIG. 1 to measure CO, SV, fluid levels, ECG waveforms, HR, arrhythmias, and motion/posture/activity level from an ambulatory patient;

FIG. 3 shows two and three-dimensional images of the sensor of FIG. 2 worn around a patient's neck;

FIG. 4 is a mechanical drawing showing a side view of the 3-part electrode and 3-part electrode holder of FIG. 1, along with an electrode spacer separating these components;

FIG. 5 is a mechanical drawing showing a top view (left-hand side) and bottom view (right-hand side) of the 3-part electrode of FIG. 1;

FIG. 6 is a mechanical drawing showing a top view of the 3-part electrode holder of FIG. 1;

FIG. 7A is a mechanical drawing showing a top view of the 3-part electrode spacer;

FIG. 7B is a mechanic drawing showing a side views of a disconnected and connected a-part electrode, electrode spacer, and electrode holder;

FIG. 8 shows a schematic drawing of electrodes used for the ECG and impedance systems positioned on the patient's chest using the sensor of FIG. 2; and,

FIG. 9 shows a schematic drawing of an electrical circuit used within the sensor of FIG. 2 to measure a TBI waveform.

### DETAILED DESCRIPTION OF THE INVENTION

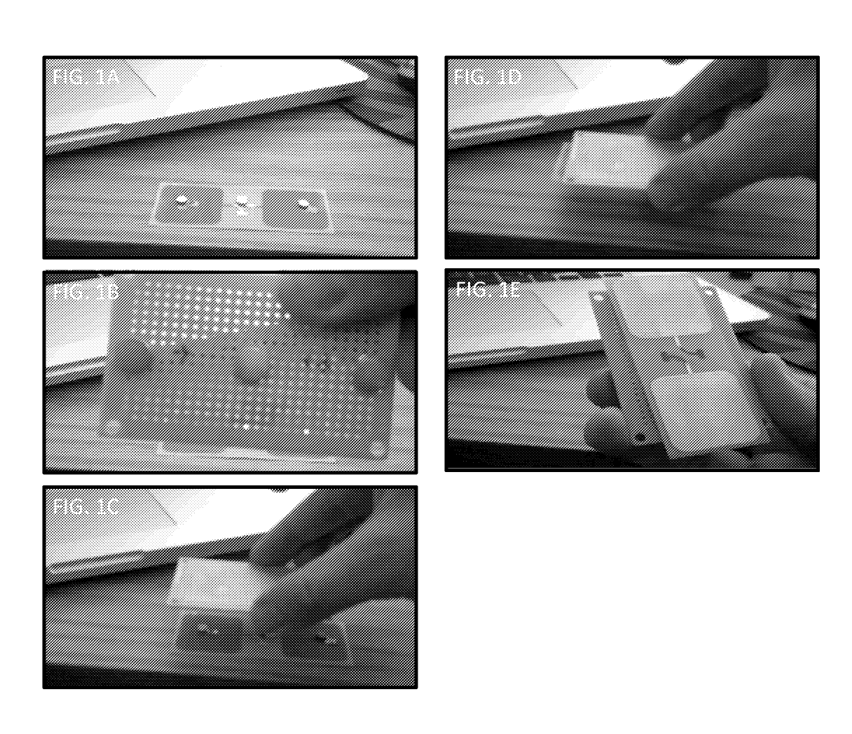

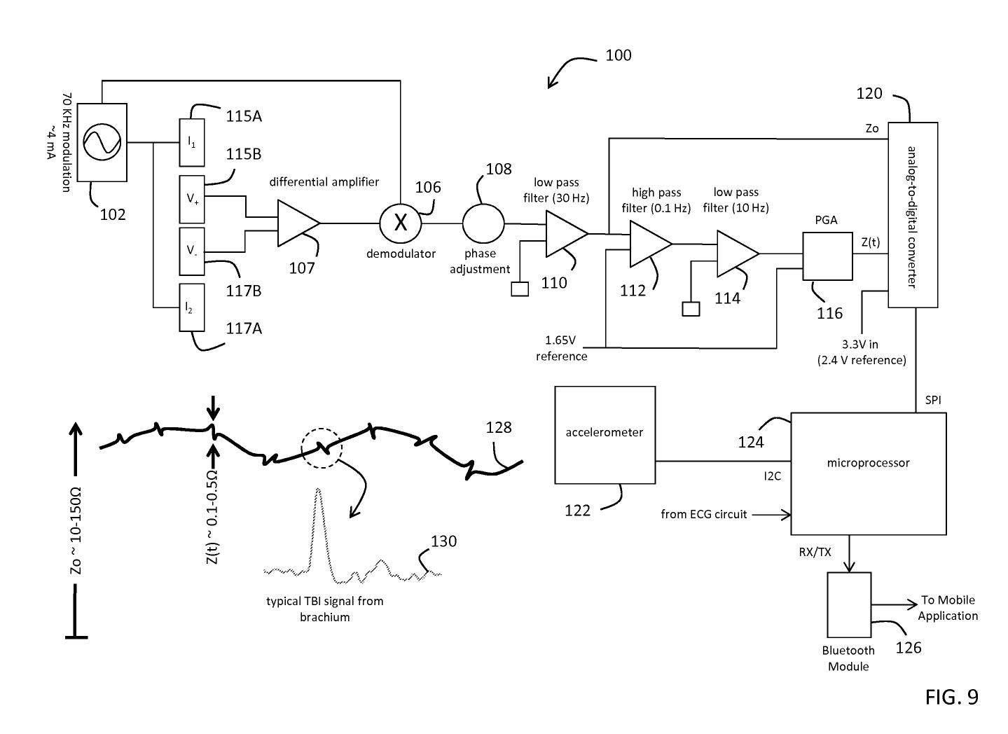

FIGS. 1A-E show time-resolved photographs of the magnetically connected electrode according to the invention. The purpose of the electrode is to simplify the mechanical/electrical connection normally used with conventional electrodes that measure physiological signals, such as those used to construct physiological waveforms (e.g. ECG or TBI waveforms). Typically, as described above, this connection is made between a metal rivet within the electrode and a mated snap within an electrical lead that connects to a circuit (e.g. an ECG circuit) used to measure the physiological waveforms. During use, the rivet is pressed into the snap to mechanically secure and electrically connect these components. In contrast, as shown in FIG. 1A, the electrode according to the invention includes three magnets on a common surface that make electrical connections with metal pads on the opposing surface. The connections, for example, are made through electrical interconnects (called ‘vias’), or simple holes punched through the electrode material (typically FR4 fiberglass). A thin film of Ag/AgCl coats the metal pads, and is then covered with a thin, conductive gel material that adheres to the skin during use. The conductive gel features electrical (e.g. electrical impedance) and mechanical properties that are similar to those of human skin. Typically the conductive gel adheres to the patient's skin when applied, and when dormant is covered with a removable material (e.g. a disposable plastic sheet) that is peeled off when the electrode is used. When combined in a vertical stack, these structures (the metal pad, Ag/AgCl film, and conductive gel) sense electrical signals from the patient that travel through wires to an electrical circuit in the sensor, such as that shown in FIG. 9. There, they are then processed as described below to measure time-dependent physiological waveforms, e.g. TBI and ECG waveforms.

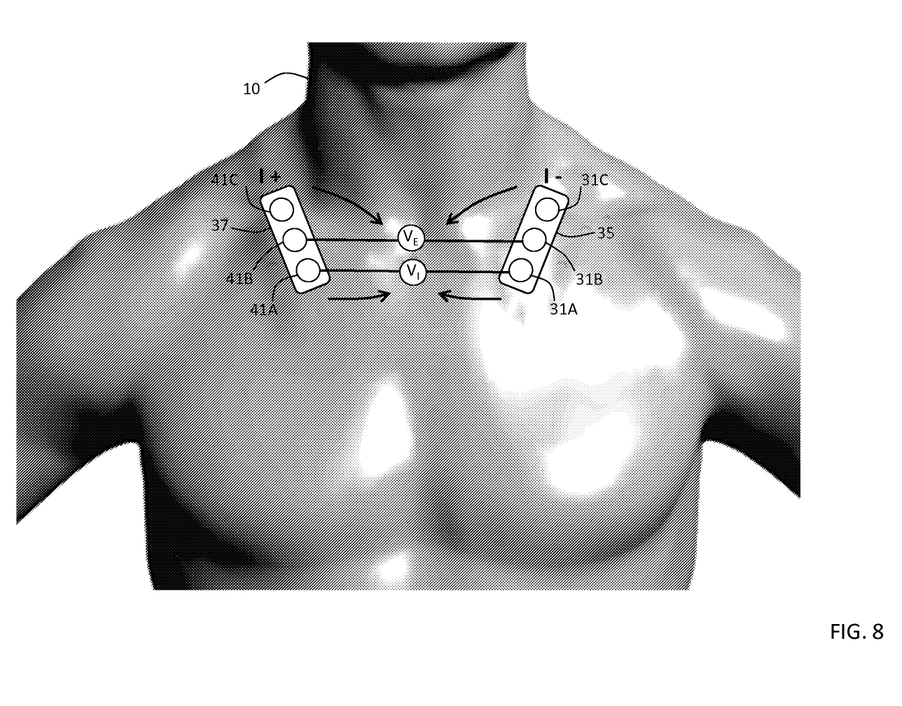

In FIG. 1A, the two magnets on the distal sides of the electrodes are used to generate TBI waveforms: one injects a high-frequency, low-amperage current, as described in detail below; the other senses a resistance that is the product of this current and the internal impedance or resistance of the patient's tissue according to Ohm's Law (V=I×R). The middle electrode is used to sense a signal that is used for ECG waveforms; techniques for this measurement are known in the art. The function of all three electrodes and their relationship to TBI and ECG waveforms is described in more detail below with regard to FIG. 8.

The magnets shown in FIG. 1A are typically permanent rare earth magnets made from alloys of rare earth materials, such as Neodymium, Boron, and Samarium Cobalt. The magnets are typically cylindrical in form and coated with a thin, conductive metal film, such as Gold, Nickel, Copper, or amalgams thereof. They are arranged on the electrode material so that they make an electrical connection with a metal pad through an underlying via. Typically they attach to the via with a conductive epoxy; rare earth magnets should not be soldered, as applied heat reduces their magnetic field. The magnets are typically arranged on the electrode material so that they each have the same pole (i.e., + or −) facing upward.

As shown in FIG. 1B, an electrode holder includes 3 matched magnets with an opposing pole to that of the magnets adhered to the electrode. These magnets could also be replaced with a magnetically active material, such as a thin film containing Iron. These magnets are geometrically oriented to align with the magnets on the electrode, and (although not shown in the photograph) make an electrical connection with analog circuits for ECG and TBI measurements, as is described in more detail below. They too are coated with a conductive metal film, such as Gold, Nickel, Copper, or amalgams thereof. Thus, when the magnets of the electrode contact the magnets of the electrode holder, an electrical connection is made between the actual electrode contacting the patient and the electrical components associated with the TBI and ECG circuits within the sensor.

Referring to FIGS. 1C and 1D, during a typical use case the electrode holder is brought proximal to the electrode, which at this point is typically not attached to the patient (i.e., the electrode and electrode holder are preferably connected before the electrode is adhered to the patient). When the electrode holder is moved to within about 1 cm of the electrode, the oppositely polled magnets generate a magnetic field that causes the electrode to quickly connect with the electrode holder, resulting in an electrical/mechanical connection between the conductive gel on the electrode and the TBI and ECG circuits connected to the electrode holder. Typically the strength of the magnetic field connecting these components is several thousand Gauss; this results in a secure connection that is similar to that provided by the typical snaps and rivets used with conventional electrodes. As shown in FIG. 1E, the magnetic field is significantly stronger than the force due to gravity, meaning the combined electrode/electrode holder structure can be picked up and then attached to the patient to make a physiological measurement.

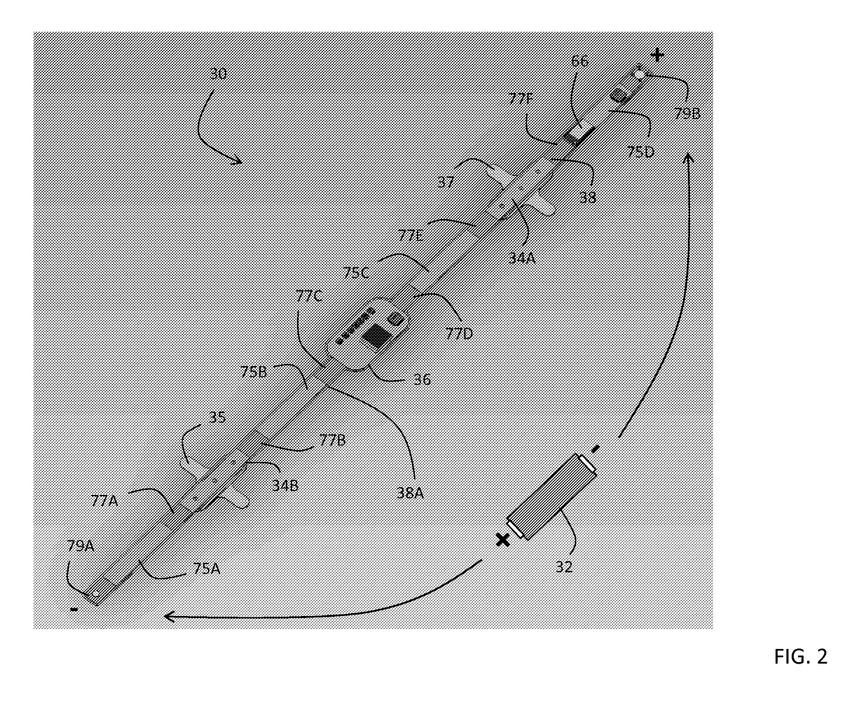

As shown in FIG. 2, the magnetically connected electrode of FIG. 1 can be used in a physiological sensor 30 that, during use, is comfortably worn around the patient's neck like a conventional necklace. In this design, the sensor's cable includes all circuit elements, which are typically distributed on an alternating combination of rigid, fiberglass circuit boards and flexible Kapton circuit boards. Typically these circuit boards are potted with a protective material, such as silicone rubber, to increase patient comfort and protect the underlying electronics. The battery for this design can be integrated directly into the cable, or connect to the cable with a conventional connector, such as a stereo-jack connector, micro-USB connector, or magnetic interface.

The sensor 30 is designed for patients suffering from CHF and other cardiac diseases, such as cardiac arrhythmias, as well as patients with implanted devices such as pacemakers and ICDs. Using the magnetically connected electrodes described herein, it makes impedance measurements to determine CO, SV, and fluid levels, and ECG measurements to determine a time-dependent ECG waveform and HR. Additionally it measures respiratory rate, skin temperature, location, and motion-related properties such as posture, activity level, falls, and degree of motion. The sensor's form factor is designed for both one-time measurements, which take just a few minutes, and continuous measurements, which can take several days. Necklaces are likely familiar to a patient 10 wearing the sensor 30, and this in turn may improve their compliance in making measurements as directed by their physician. Ultimately compliance in using the sensor may improve the patient's physiological condition. Moreover, the sensor is designed to make measurements near the center of the chest, which is relatively insensitive to motion compared to distal extremities, like the arms or hands. The sensor's form factor also ensures relatively consistent electrode placement for the impedance and ECG measurements; this is important for one-time measurements made on a daily basis, as it minimizes day-to-day errors associated with electrode placement. Finally, the sensor's form factor distributes electronics around the patient's neck, thereby minimizing bulk and clutter associated with these components and making the sensor 30 more comfortable to the patient.

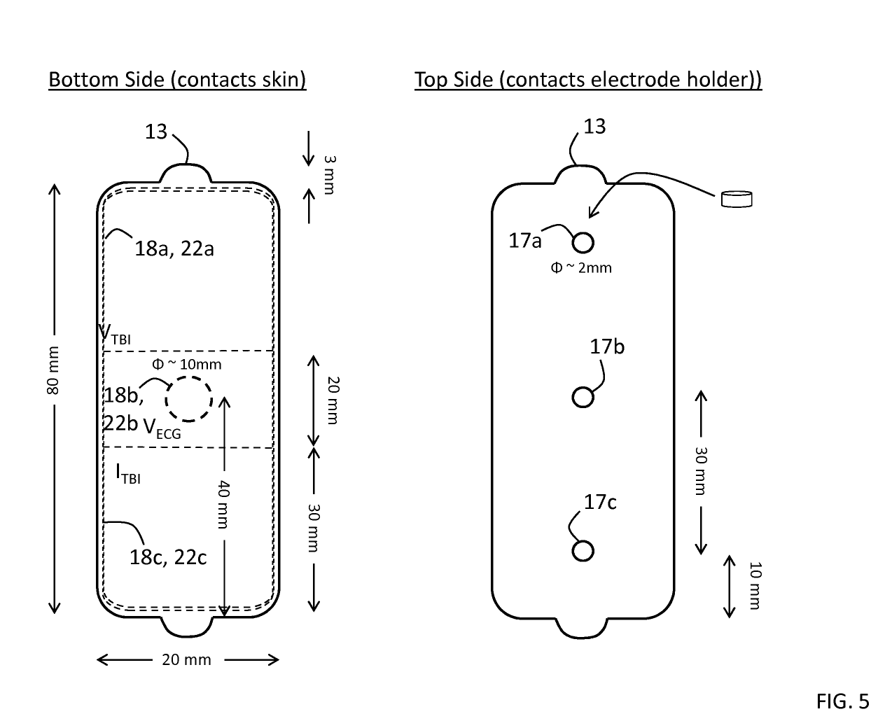

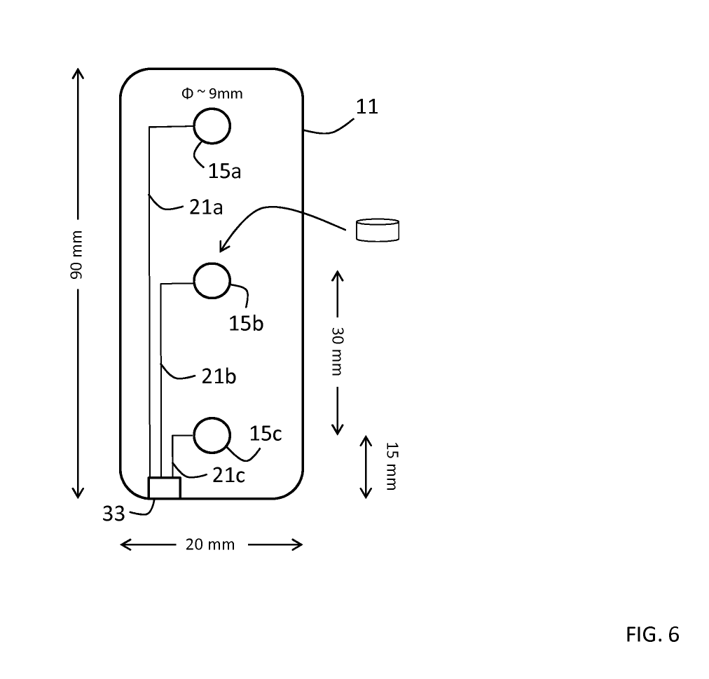

In one embodiment the sensor 30 features a pair of electrode holders 34A, 34B, located on opposing sides of the necklace, that each include magnets as described in more detail with respect to FIG. 6. The electrode holders 34A, 35A each receive a separate 3-part magnetically connected electrode patch 35, 37, described in more detail with respect to FIG. 5. During use, the electrode patches 35, 37 connect to their respective electrode holders 34A, 34B through the magnetic interface, and then stick to the patient's chest when the sensor 30 is draped around their neck. An adhesive backing supports each conductive electrode within the electrode patch 35, 37. The electrodes feature a sticky, conductive gel that contacts the patient's skin. The conductive gel contacts a metal pad that is coated on one side with a thin layer of Ag/AgCl, and connects to a magnet through a via. As described in more detail with respect to FIG. 8, the outer electrodes in each electrode patch are used for the impedance measurement (they conduct signals V+/−, I+/−), while the inner electrodes are used for the ECG measurement (they conduct signals ECG+/−). Proper spacing of the electrodes ensures both impedance and ECG waveforms having high signal-to-noise ratios; this in turn leads to measurements that are relatively easy to analyze, and thus have optimum accuracy. FIG. 5 shows preferred dimensions for these components.

A flexible, flat cable 38 featuring a collection of conductive members transmits signals from the electrode patches 35, 37 to an electronics module 36, which, during use, is preferably worn near the back of the neck. Typically the cable 38 includes alternating regions of rigid fiberglass circuit boards 75A-D and flexible Kapton flex circuits 77A-F to house other electronic components (used, e.g., for other measurement circuits) and conduct electrical signals. The electronic module 36 may snap into a soft covering to increase comfort. The electronics module 36 features a first electrical circuit for making an impedance-based measurement of TBI waveforms that yield CO, SV, and fluid levels, and a second electrical circuit for making differential voltage measurements of ECG waveforms that yield HR and arrhythmia information. The first electrical circuit, which is relatively complex, is shown schematically in FIG. 9; the second electrical circuit is well known in this particular art, and is thus not described in detail here.

During a measurement, the second electrical circuit measures an analog ECG waveform that is received by an internal analog-to-digital converter within a microprocessor. The microprocessor analyzes this signal to simply determine that the electrode patches are properly adhered to the patient, and that the system is operating satisfactorily. Once this state is achieved, the first and second electrical circuits generate time-dependent analog waveforms that a high-resolution analog-to-digital converter within the electronics module 36 receives and then sequentially digitizes to generate time-dependent digital waveforms. Analog waveforms can be switched over to this component, for example, using a field effect transistor (FET). Typically these waveforms are digitized with 16-bit resolution over a range of about −5V to 5V. The microprocessor receives the digital waveforms and processes them with computational algorithms, written in embedded computer code (such as C or Java), to generate values of CO, SV, fluid level, and HR. Additionally, the electronics module 36 features a 3-axis accelerometer and temperature sensor to measure, respectively, three time-dependent motion waveforms (along x, y, and z-axes) and temperature values. The microprocessor analyzes the time-dependent motion waveforms to determine motion-related properties such as posture, activity level, falls, and degree of motion. Temperature values indicate the patient's skin temperature, and can be used to estimate their core temperature (a parameter familiar to physicians), as well as ancillary conditions, such as perfusion, ambient temperature, and skin impedance. Motion-related parameters are determined using techniques known in the art. Temperature values are preferably reported in digital form that the microprocessor receives through a standard serial interface, such as I2C, SPI, or UART.

Both numerical and waveform data processed with the microprocessor are ported to a wireless transmitter 66, such as a transmitter based on protocols like Bluetooth or 802.11a/b/g/n. From there, the transmitter sends data to an external receiver, such as a conventional cellular telephone, tablet, wireless hub (such as Qualcomm's 2Net system), or personal computer. Devices like these can serve as a ‘hub’ to forward data to an Internet-connected remote server located, e.g., in a hospital, medical clinic, nursing facility, or eldercare facility.

Referring back to FIG. 2, a battery module 32 featuring a rechargeable Li:ion battery connects at two points to the cable 38 using a pair of connectors 79A, 79B. During use, the connectors 79A, 79B plug into a pair of mated connectors on the battery module 32 that securely hold the terminal ends of the cable 38 so that the sensor 30 can be comfortably and securely draped around the patient's neck. Importantly, when both connectors 79A, 79B are plugged into the battery module 32, the circuit within the sensor 30 is completed, and the battery module 32 supplies power to the electronics module 36 to drive the above-mentioned measurements. The connectors 79A, 79B terminating the cable can also be disconnected from the connectors on the battery module 32 so that this component can be replaced without removing the sensor 30 from the patient's neck. Replacing the battery module 32 in this manner means the sensor 30 can be worn for extended periods of time without having to remove it from the patient. In general, the connectors 79A, 79B can take a variety of forms: they can be flat, multi-pin connectors, magnetic connectors, or stereo-jack type connectors that quickly plug into a female adaptor. Typically an LED on the battery module indicates that this is the case, and that the system is operational. When the battery within battery module 32 is nearly drained, the LED indicates this particular state (e.g., by changing color, or blinking periodically). This prompts a user to unplug the battery module 32 from the two connectors, plug it into a recharge circuit (not shown in the figure), and replace it with a fresh battery module as described above.

As is clear from FIG. 2, the neck-worn cable 38 serves four distinct purposes: 1) it transfers power from the battery module 32 to the electronics module 36; 2) it ports signals from the electrode patches 35, 37 to the impedance and ECG circuits; 3) it ensures consistent electrode placement for the impedance and ECG measurements to reduce measurement errors; and 4) it distributes the various electronics components and thus allows the sensor to be comfortably worn around the patient's neck. Typically each arm of the cable 38 will have six wires: two for the impedance electrodes, one for the ECG electrode, and three to pass signals from the electronics module to electrical components within the battery module. These wires can be included as discrete elements, a flex circuit, or, as described above, a flexible cable.

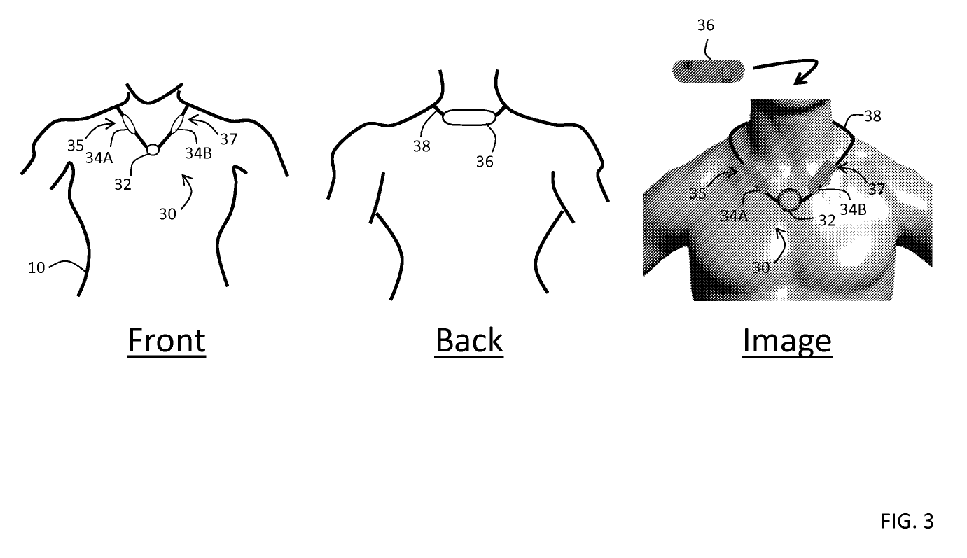

FIG. 3 shows the above-described sensor 30 worn around the neck of a patient 10. As described above, the sensor 30 includes an electronics module 36 worn on the back of the patient's neck, a battery module 32 in the front, and electrode holders 34A, 34B that connect to the magnetically active electrode patches 35, 37 and secure the cable 38 around the patient's neck that make impedance and ECG measurements.

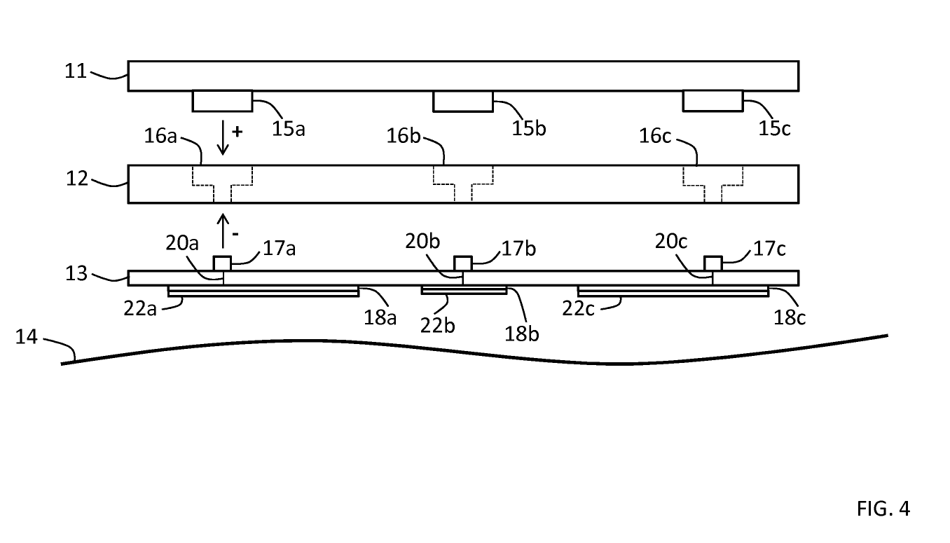

FIGS. 4-7 show a more detailed view of the magnetically connected electrode 13, electrode holder 11, and electrode spacer 12 described above. As shown in the figures, the electrode 13 features three electrode regions, each with a conductive gel 22a-c, metal pad coated with an Ag/Ag/Cl film 18a-c, magnet 17a-c, and a via 20a-c (i.e., an electrical interconnect) that provides an electrical connection between the metal pad coated with the Ag/Ag/Cl film 18a-c. The electrode 13 includes three electrode regions, and is designed to integrate with the neck-worn sensor shown in FIG. 2. However, this same design could be used for electrodes just having any number of electrode regions, in particular a single electrode region, e.g. those used for conventional ECG electrodes.

Referring again to FIGS. 4-7, during use the conductive gel 22a-c of each electrode region adheres to a patient's skin 14. Typically, as described above, when the electrode 13 is not in use, the conductive gel 22a-c is covered with a thin, disposable plastic film (not shown in the figure) that keeps the gel 22a-c moist and preserves its adhesive properties. Each magnet 17a-c in the electrode 13 is oriented so that the same pole is pointing upward; FIG. 4 shows this pole as ‘-’.

An electrode holder 11 includes three larger magnets 15a-c that are geometrically aligned with the magnets 17a-c in the electrode. The poles of the larger magnets 15a-c within the electrode holder 11 oppose those of the magnets 17a-c attached to the electrode; FIG. 4 shows this pole as ‘+’. As described above, both the magnets 17a-c of the electrode 13 and the magnets 15a-c of the electrode holder 11 are coated with an electrically conductive metal film. Thus, when they come in contact, electricity can flow from one magnet to the other.

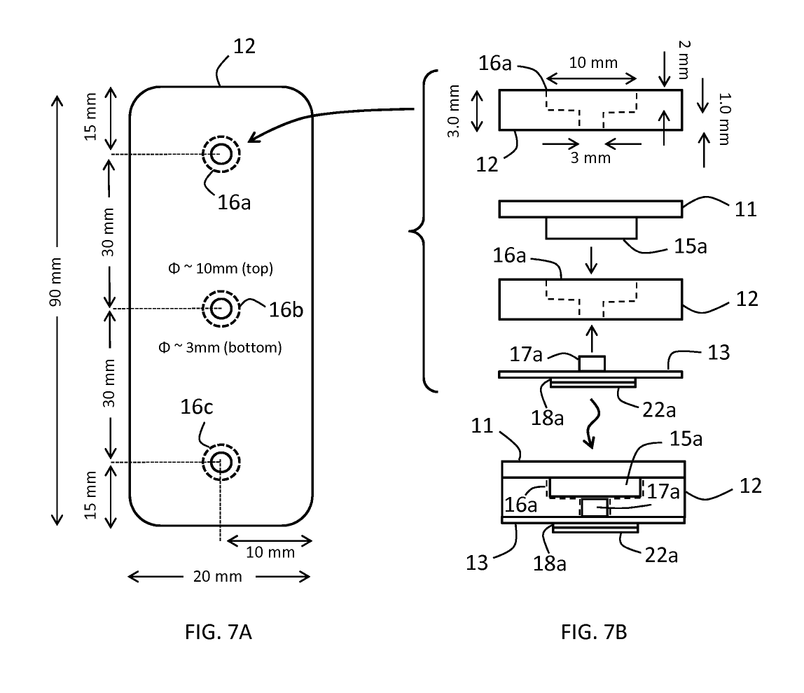

An electrode spacer 12 separates the electrode 13 from the electrode holder 11. The electrode spacer 12 is typically made from an electrically insulating material, such as molded ABS plastic, nylon, or Delrin. It features three separate countersunk holes 16a-c that, during use, accommodate the magnets 17a-c from the electrode 13 and those 15a-c from the electrode holder 11. The electrode spacer 12 separates the electrode 13 and electrode holder 11 during use, and ensures that magnets within these components align and make good electrical contact during a measurement. FIG. 7, and particularly the images shown on the right-hand side, indicates how each of these components fit together.

FIG. 5 shows a more detailed view of the electrode 13. As described above, when used with the sensor shown in FIG. 1, the electrode features 3 individual electrode regions that each include an Ag/AgCl-coated metal film 18a-c, and an overlying conductive gel 22a-c. To increase the signal-to-noise ratio of the relatively weak TBI waveform, the electrodes used for this measurement (labeled V_{TBI} and I_{TBI}) are positioned distally and have a relatively large surface are compared to the central electrode used for the ECG measurement (labeled V_{ECG}). As shown in the figure, the TBI electrodes each have an area of about 30 mm×20 mm and are typically square in shape; the ECG electrode is typically round in shape, and has a diameter of about 10 mm. There should be a spacing of about 20 mm between the TBI electrodes to avoid any signal distortion or cross-talk between the electrodes. Typically the magnets located on the opposite side of the electrode materials have a diameter of about 2 mm, a height of about 1 mm, and, as described above, connect to the underlying electrode materials using underlying vias.

FIG. 6 shows a top view of the electrode holder 11. This component typically includes a larger magnet 15a-c (preferably 9 mm in diameter and a height of 2 mm) than that used in the electrode 13. The larger magnet 15a-c increases the magnetic field and resultant attraction force between the electrode 13 and the electrode holder 11, and means that a small, relatively low-cost magnet can be used in the electrode 13. This is desirable, given this component is typically disposable, and thus it is paramount to reduce its cost. The electrode holder 11 features electrical traces 21a-c that connect the magnets 15a-c to a bulkhead connector 33 located at the holder's distal end. That bulkhead connector 33 connects to a mated connector (not shown in the figure) that, during a measurement, ports electrical signals measured by the electrodes to the TBI and ECG analog circuits for processing.

FIG. 7A shows a top view of the electrode spacer 11 and its preferred dimensions. FIG. 7B indicates how the electrode 13, electrode holder 11, and electrode spacer 12, along with all the ancillary components described above, fit together during a physiological measurement.

FIG. 8 indicates in more detail how the above-described electrode measures TBI waveforms and CO/SV values from a patient. As described above, 3-part electrode patches 35, 37 within the neck-worn sensor attach to the patient's chest. Ideally, each patch 35, 37 attaches just below the collarbone near the patient's left and right arms. During a measurement, the impedance circuit injects a high-frequency, low-amperage current (I) through outer electrodes 31C, 41C. Typically the modulation frequency is about 70 kHz, and the current is about 4 mA. The current injected by each electrode 31C, 41C is out of phase by 180°. It encounters static (i.e. time-independent) resistance from components such as bone, skin, and other tissue in the patient's chest. Additionally, blood and fluids in the chest conduct the current to some extent. Blood ejected from the left ventricle of the heart into the aorta, along with fluids accumulating in the chest, both provide a dynamic (i.e. time-dependent) resistance. The aorta is the largest artery passing blood out of the heart, and thus it has a dominant impact on the dynamic resistance; other vessels, such as the superior vena cava, will contribute in a minimal way to the dynamic resistance.

Inner electrodes 31A, 41A measure a time-dependent voltage (V) that varies with resistance (R) encountered by the injected current (I). This relationship is based on Ohm's Law, as described above. During a measurement, the time-dependent voltage is filtered by the impedance circuit, and ultimately measured with an analog-to-digital converter within the electronics module. This voltage is then processed to calculate SV with an equation such as that shown below in Eq. 3, which is Sramek-Bernstein equation, or a mathematical variation thereof. Historically parameters extracted from TBI signals are fed into the equation, shown below, which is based on a volumetric expansion model taken from the aortic artery:

$$S V = δ \frac{{L}^{3}}{4.25} \frac{{( d Z ( t ) / d t )}_{max}}{{Z}_{0}} L V E T \qquad ( 3 )$$

In Eq. 3, Z(t) represents the TBI waveform, δ represents compensation for body mass index, Zo is the base impedance, L is estimated from the distance separating the current-injecting and voltage-measuring electrodes on the thorax, and LVET is the left ventricular ejection time, which can be determined from the TBI waveform, or from the HR using an equation called ‘Weissler's Regression’, shown below in Eq. 4, that estimates LVET from HR:

LVET=−0.0017×HR+0.413 (4)

Weissler's Regression allows LVET, to be estimated from HR determined from the ECG waveform. This equation and several mathematical derivatives, along with the parameters shown in Eq. 3, are described in detail in the following reference, the contents of which are incorporated herein by reference: Bernstein, Impedance cardiography: Pulsatile blood flow and the biophysical and electrodynamic basis for the stroke volume equations; J Electr Bioimp; 1: 2-17 (2010). Both the Sramek-Bernstein Equation and an earlier derivative of this, called the Kubicek Equation, feature a ‘static component’, Z₀, and a ‘dynamic component’, ΔZ(t), which relates to LVET and a (dZ/dt)ₘₐₓ/Zₒ value, calculated from the derivative of the raw TBI signal, Z(t). These equations assume that (dZ(t)/dt)ₘₐₓ/Zₒ represents a radial velocity (with units of Ω/s) of blood due to volume expansion of the aorta.

In Eq. 3 above, the parameter Z₀ will vary with fluid levels. Typically a high resistance (e.g. one above about 30Ω) indicates a dry, dehydrated state. Here, the lack of conducting thoracic fluids increases resistivity in the patient's chest. Conversely, a low resistance (e.g. one below about 19Ω) indicates the patient has more thoracic fluids, and is possibly overhydrated. Here, the abundance of conducting thoracic fluids decreases resistivity in the patient's chest. The TBI circuit and specific electrodes used for a measurement may affect these values. Thus, the values can be more refined by conducting a clinical study with a large number of subjects, preferably those in various states of CHF, and then empirically determining ‘high’ and ‘low’ resistance values.

FIG. 9 shows an analog circuit 100 that performs the impedance measurement according to the invention. The figure shows just one embodiment of the circuit 100; similar electrical results can be achieved using a design and collection of electrical components that differ from those shown in the figure.

The circuit 100 features a first magnetically connected electrode 115A that injects a high-frequency, low-amperage current (I₁) into the patient's brachium. This serves as the current source. Typically a current pump 102 provides the modulated current, with the modulation frequency typically being between 50-100 KHz, and the current magnitude being between 0.1 and 10 mA. Preferably the current pump 102 supplies current with a magnitude of 4 mA that is modulated at 70 kHz through the first electrode 115A. A second magnetically connected electrode 117A injects an identical current (I₂) that is out of phase from I₁ by 180°.

Another pair of magnetically connected electrodes 115B, 117B measure the time-dependent voltage encountered by the propagating current. These electrodes are indicated in the figure as V+ and V−. As described above, using Ohm's law, the measured voltage divided by the magnitude of the injected current yields a time-dependent resistance to ac (i.e. impedance) that relates to blood flow in the aortic artery. As shown by the waveform 128 in the figure, the time-dependent resistance features a slowly varying dc offset, characterized by Zo, that indicates the baseline impedance encountered by the injected current; for TBI this will depend, for example, on the amount of thoracic fluids, along with the fat, bone, muscle, and blood volume in the chest of a given patient. Zo, which typically has a value between about 10 and 150Ω, is also influenced by low-frequency, time-dependent processes such as respiration. Such processes affect the inherent capacitance near the chest region that TBI measures, and are manifested in the waveform by low-frequency undulations, such as those shown in the waveform 128. A relatively small (typically 0.1-0.5Ω) AC component, ΔZ(t), lies on top of Zo and is attributed to changes in resistance caused by the heartbeat-induced blood that propagates in the brachial artery, as described in detail above. Z(t) is processed with a high-pass filter to form a TBI signal that features a collection of individual pulses 130 that are ultimately processed to ultimately determine SV and CO.

Voltage signals measured by the first electrode 115B (V+) and the second electrode 117B (V−) feed into a differential amplifier 107 to form a single, differential voltage signal which is modulated according to the modulation frequency (e.g. 70 kHz) of the current pump 102. From there, the signal flows to a demodulator 106, which also receives a carrier frequency from the current pump 102 to selectively extract signal components that only correspond to the TBI measurement. The collective function of the differential amplifier 107 and demodulator 106 can be accomplished with many different circuits aimed at extracting weak signals, like the TBI signal, from noise. For example, these components can be combined to form a ‘lock-in amplifier’ that selectively amplifies signal components occurring at a well-defined carrier frequency. Or the signal and carrier frequencies can be deconvoluted in much the same way as that used in conventional AM radio using a circuit that features one or more diodes. The phase of the demodulated signal may also be adjusted with a phase-adjusting component 108 during the amplification process. In one embodiment, the ADS 1298 family of chipsets marketed by Texas Instruments may be used for this application. This chipset features fully integrated analog front ends for both ECG and impedance pneumography. The latter measurement is performed with components for digital differential amplification, demodulation, and phase adjustment, such as those used for the TBI measurement, that are integrated directly into the chipset.

Once the TBI signal is extracted, it flows to a series of analog filters 110, 112, 114 within the circuit 100 that remove extraneous noise from the Zo and ΔZ(t) signals. The first low-pass filter 110 (30 Hz) removes any high-frequency noise components (e.g. power line components at 60 Hz) that may corrupt the signal. Part of this signal that passes through this filter 110, which represents Zo, is ported directly to a channel in an analog-to-digital converter 120. The remaining part of the signal feeds into a high-pass filter 112 (0.1 Hz) that passes high-frequency signal components responsible for the shape of individual TBI pulses 130. This signal then passes through a final low-pass filter 114 (10 Hz) to further remove any high-frequency noise. Finally, the filtered signal passes through a programmable gain amplifier (PGA) 116, which, using a 1.65V reference, amplifies the resultant signal with a computer-controlled gain. The amplified signal represents ΔZ(t), and is ported to a separate channel of the analog-to-digital converter 120, where it is digitized alongside of Zo. The analog-to-digital converter and PGA are integrated directly into the ADS 1298 chipset described above. The chipset can simultaneously digitize waveforms such as Zo and ΔZ(t) with 24-bit resolution and sampling rates (e.g. 500 Hz) that are suitable for physiological waveforms. Thus, in theory, this one chipset can perform the function of the differential amplifier 107, demodulator 108, PGA 116, and analog-to-digital converter 120. Reliance of just a single chipset to perform these multiple functions ultimately reduces both size and power consumption of the TBI circuit 100.

Digitized Zo and Z(t) waveforms are received by a microprocessor 124 through a conventional digital interface, such as a SPI or I2C interface. Algorithms for converting the waveforms into actual measurements of SV and CO are performed by the microprocessor 124. The microprocessor 124 also receives digital motion-related waveforms from an on-board accelerometer, and processes these to determine parameters such as the degree/magnitude of motion, frequency of motion, posture, and activity level.

In other embodiments, the necklace-shaped sensor described above can be augmented to include other physiological sensors, such as a pulse oximeter or blood pressure monitor. For example, the pulse oximetry circuit can be included on a rigid circuit board within the necklace, and then can connect to an ear-worn oximetry sensor. The geometry of the sensor described herein, and its proximity to the patient's ear, makes this measurement possible. For blood pressure, a parameter called pulse transit time, which is measured between a fiducial point on the ECG waveform (e.g. the QRS complex) and a fiducial point (e.g. an onset) of a TBI pulse or photoplethysmogram measured by the pulse oximeter, correlates inversely to blood pressure. Thus measuring this parameter and calibrating it with a conventional measurement of blood pressure, such as that done with an oscillometric cuff, can yield a continuous, non-invasive measurement of blood pressure.

Still other embodiments are within the scope of the following claims.

What is claimed is:

1. A method for measuring a bioimpedance signal from a patient, comprising:
contacting a first region of the patient's body with a first electrode, the first electrode comprising: i) a first conductive gel: ii) a first Ag/AgCl film comprising Ag/AgCl in electrical contact with the first conductive gel; iii) a first metal film in electrical contact with the first Ag/AgCl film; and iv) a first magnet in electrical contact with the first metal film;
injecting an electrical current into the patient through the first magnet and into the first electrode;
contacting a second region of the patient's body with a second electrode, the second electrode comprising: i) a second conductive gel: ii) a second Ag/AgCl film comprising Ag/AgCl in electrical contact with the second conductive gel; iii) a second metal film in electrical contact with the second Ag/AgCl film; and iv) a second magnet in electrical contact with the second metal film;
measuring a signal from the second magnet to generate a voltage, the voltage related to a product of the electrical current injected through the first electrode and a bioimpedance of the patient; and,
processing the voltage to generate the bioimpedance signal.
